(19) United States
(12) Patent Application Publication
(10) Pub. No.: US 2025/0255798 A1
(43) Pub. Date: Aug. 14, 2025

(54) HYDROGELS FOR SOFT TISSUE FILLING

(71) Applicant: TEOXANE SA, GENEVE (CH)

(72) Inventors: Jimmy FAIVRE, VALSERHONE (FR); Elodie CLERC, FILLIERE (FR); François BOURDON, GAILLARD (FR)

(21) Appl. No.: 18/857,031

(22) PCT Filed: Apr. 14, 2023
(86) PCT No.: PCT/EP2023/059831

§ 371 Date: Oct. 15, 2024

(30) Foreign Application Priority Data
Apr. 15, 2022 (FR) FR2203541

(51) Int. Cl. A61K 8/73; A61Q 19/08; C08B 37/08; C08J 3/075; C08J 3/24

(52) U.S. Cl. CPC A61K 8/735; A61Q 19/08; C08B 37/0072; C08J 3/075; C08J 3/24; A61K 2800/91

(57) ABSTRACT

The present disclosure relates to a method for preparing a hydrogel comprising a crosslinked polysaccharide, in particular, a process for preparing an injectable hydrogel comprising crosslinked hyaluronic acid. The present disclosure also relates to a hydrogel, preferably injectable, obtainable by the process, a composition comprising the hydrogel, and uses of the hydrogel.

### FIELD OF THE INVENTION

The present invention concerns a method for preparing a hydrogel comprising a crosslinked polysaccharide, in particular, a method for preparing an injectable hydrogel comprising crosslinked hyaluronic acid. The present invention also concerns a hydrogel, preferably injectable, that can be obtained by the method, a composition comprising the hydrogel, and the uses of this hydrogel.

### TECHNOLOGICAL BACKGROUND

Polysaccharide-based hydrogels, in particular hyaluronic acid-based hydrogels, are widely used for soft tissues filling, in particular the skin, which have, for example, volume defects (e.g., wrinkles, scars) or for increasing their volume. Hydrogels suitable for soft tissue filling are typically crosslinked hydrogels, i.e., the polysaccharide is crosslinked by means of one or more cross-linking agents. This crosslinking makes it possible to obtain a hydrogel with the mechanical properties desirable for soft tissues filling.

Although biodegradable and biocompatible, hyaluronic acid-based hydrogels can cause adverse reactions, with a low incidence, that develop between 6 and 24 months after application of the filler. In particular, it may happen that the filler migrates to a zone far from that of its application in or on the body (Chae S Y, Lee K C, Jang Y H, Lee S J, Kim D W, Lee W J. A Case of the Migration of Hyaluronic Acid Filler from Nose to Forehead Occurring as Two Sequential Soft Lumps. Ann Dermatol. 2016; 28(5):645-647 and Mosleh, Rasha & Mukari, Abed & Krausz, Judit & Hartstein, Morris & Hamed Azzam, Shirin. (2019). Orbit mass secondary to migration of dermal hyaluronic acid filler. JAAD Case Reports. 5. 488-490). Several reasons have been proposed to explain this phenomenon, such as, for example, a poor injection technique, too large a volume injected, post-injection physical activity (muscle activity, exercise), displacement induced by pressure (e.g., massage), intravascular injection, lymphatic invasion or gravity. To reduce the risk of migration of polysaccharide-based gels, certain methods have already been described; it has especially been proposed to apply a repellent electric field to at least part of the perimeter around the treatment site (WO2020/172371).

Nevertheless, there is still a need to provide a hydrogel based on crosslinked polysaccharides, in particular a hydrogel based on crosslinked hyaluronic acid whose the hyaluronic acid chains are preserved, having mechanical properties suitable for soft tissue filling and which does not migrate in tissues after application.

### BRIEF DESCRIPTION OF THE INVENTION

The present invention relates to a method for preparing a hydrogel, preferably injectable, comprising the following steps:
a) providing at least one polysaccharide;
b) providing at least one crosslinking agent, said cross-linking agent comprising at least two functional groups Z, identical or different, chosen from isocyanate, amino, epoxide, carboxyl, N-succinimidyloxycarbonyl, N-sulfosuccinimidyloxycarbonyl, halocarbonyl, isothiocyanate, vinyl, formyl, hydroxyl, sulfhydryl, hydrazino, acylhydrazino, aminoxy, carbodiimide, and an acid anhydride residue;
c) preparing a crosslinking reaction medium comprising the polysaccharide(s), the cross-linking agent(s) and a solvent, the total quantity of cross-linking agent ranging from 0.001 to less than 0.02 mole per 1 mole of polysaccharide repeating unit, the duration of the reaction medium preparation step not exceeding 5 hours;
d) placing the reaction medium obtained at the end of step c), for a period ranging from 2 weeks to 17 weeks, at a pressure P less than or equal to atmospheric pressure and at a temperature T greater than the eutectic point temperature of the reaction medium as measured at pressure P and less than the freezing point temperature of the reaction medium as measured at pressure P.

The present invention also relates to a hydrogel capable of being obtained by a method as described herein, as well as to a cosmetic or pharmaceutical composition comprising such a hydrogel and their uses in filling and/or replacing tissues, for preventing and/or treating the change of viscoelastic or biomechanical properties of the skin; for filling in volume defects of the skin, and especially for filling wrinkles, fine lines and scars; for example, to reduce nasolabial folds and frown lines, to increase the volume of the cheekbones, chin or lips, to restore facial volume, especially the cheeks, temples, contour of the face and area around the eyes; or to reduce the appearance of wrinkles and fine lines; or to stimulate, regenerate, moisturize, firm or restore the radiance of the skin, especially by mesotherapy.

Other aspects of the invention are as described below and in the claims.

### FIGURES

FIG. 1 illustrates the stickiness of a hydrogel according to the invention compared to a conventional hydrogel on a metal (FIG. 1A), glass (FIG. 1B), plastic (FIG. 1C) and collagen (FIG. 1D) surface.

### DEFINITIONS

The term “gel” designates a network of polymers which is expanded throughout its volume by a fluid. This means that a gel consists of two media, one solid and the other liquid, dispersed in one another. The so-called solid medium is made up of long polymer molecules connected together by weak bonds (for example hydrogen bonds) or by covalent bonds (cross-linking). The liquid medium is made up of a solvent. A gel generally corresponds to a product which has a phase angle δ of less than or equal to 45° at 1 Hz for a deformation of 0.1% or a pressure of 1 Pa, advantageously a phase angle δ ranging from 2° to 45° or ranging from 20° to 45°.

The term “hydrogel” designates a gel as defined above in which the solvent constituting the liquid medium is predominantly water (for example at least 90%, in particular at least 95%, especially at least 99% by weight of the liquid medium). Preferably, the liquid medium comprises, especially consists of, a buffer solution, advantageously allowing a pH of the liquid medium comprised between 6.8 and 7.8, especially a saline phosphate buffer.

The term “injectable gel” designates a gel which can flow and be injected manually by means of a syringe equipped with a needle of diameter ranging from 0.1 to 0.5 mm, for example a hypodermic needle of 30 G, 27 G, 26 G or 25 G. Preferentially, an injectable gel is a gel having an average extrusion force of less than or equal to 25 N, preferably ranging from 5 to 25 N, more preferably ranging from 8 to 15 N, when measured with a dynamometer, at a fixed speed of approximately 12.5 mm/min, in syringes of external diameter less than or equal to 6.3 mm, with a needle of diameter less than or equal to 0.4 mm (27 G) and length of % inch, at room temperature.

The extensibility of a product refers to its ability to be stretched between two surfaces to which it has adhered. The extensibility can be determined using a texture analyzer, a sensory analysis performed by a panel, or rheological and mechanical measurements especially including the measurement of the phase angle (δ) or tensile tests. In particular, this characteristic can be measured as described by P. Micheels et al. (Micheels et al., Comparison of two Swiss-designed hyaluronic acid gels: Six-month clinical follow-up, Journal of Drugs in Dermatology, 2017, 16:154-161, “Resistance to stretching”) or by carrying out a tack test and measuring the length of the gel threads in traction.

The stickiness of a product refers to its ability to adhere to a surface. It can be qualitatively determined using a sensory analysis performed using a panel or by moving a bolus on a surface. It can also be quantitatively determined by measuring the adhesion force to a surface, by a traction machine or by mechanical analysis.

The term “polysaccharide” designates a polymer composed of monosaccharides (preferentially D enantiomers) joined together by glycosidic bonds.

The term “monosaccharide” designates an unmodified or modified monosaccharide.

An “unmodified monosaccharide” means a compound of the formula H—(CHOH)ₓ—CO—(CHOH)_{y}—H where x and y are independently an integer from 0 to 5 with the condition that 2≤x+y≤5, the monosaccharide may be in a linear form represented by the above formula or may be in a cyclized form by reaction of the CO function (aldehyde or ketone) with one of the OH groups to form a hemiacetal or hemiketal group. Preferably, the monosaccharide is in cyclized form. There are two types of monosaccharides: aldoses that carry an aldehyde function (when x or y is 0) and ketoses that carry a ketone function (when neither x nor y is 0). Monosaccharides are classified by number of carbons. For example, the monosaccharides with 6 carbons (x+y=5) are hexoses of formula C₆H₁₂O₆ and can be allose, altrose, glucose, mannose, gulose, idose, galactose or talose. Monosaccharides with 5 carbons (x+y=4) are pentoses of formula C₅H₁₀O₅ and can be ribose, arabinose, xylose, or lyxose. Preferably, the monosaccharide is hexose, i.e. x+y=5. A monosaccharide further comprises x+y asymmetric carbons and thus 2^{(x+y-1)} pairs of enantiomers. Each pair of enantiomers is designated by a different name and the enantiomers of the same pair are referred to as D and L enantiomers, respectively.

A “modified monosaccharide” designates an unmodified monosaccharide as defined above, for example:
one or more of the OH functional groups have been replaced by another functional group, for example:
(i) an OR group with R representing a (C₁-C₆) alkyl group such as methyl or ethyl; hydroxy-(C₁-C₆)alkyl such as hydroxyethyl (—CH₂CH₂OH) or hydroxypropyl (—CH₂—CH(OH)—CH₃); carboxy-(C₁-C₆)alkyl such as carboxymethyl (—CH₂COOH); or CO—(C₁-C₆)alkyl such as acetyl; and/or/or
(ii) an NR′R″ group with R′ and R″ independently representing H, (C₁-C₆)alkyl or CO—(C₁-C₆)alkyl such as acetyl; and/or
(iii) an OSO₃H group; and/or
   the terminal CH₂OH functional group(s) have been replaced by a COOH or CHO group; a —CH(OH)—CH(OH)—bond is oxidized to give two-terminal CHO (aldehyde) groups in place of this bond; and/or
   a terminal CH₂OH function was fused with an OH functional group to form an —O—CH₂-chain.

The expression “repeating unit” of a polysaccharide designates a structural unit made up of one or more (usually 1 or 2) monosaccharides whose repetition produces the complete polysaccharide chain.

Some or all of the monosaccharides may be in modified form.

The monosaccharides, when modified, can be in different modified forms.

The term “physiologically acceptable” designates something that is generally safe, non-toxic and neither biologically nor otherwise undesirable and that is acceptable for cosmetic (i.e. non-therapeutic) or therapeutic human or veterinary use, including for use by injection into the human or animal body or for topical application to the skin.

The salts useful in the context of the present invention are preferably physiologically acceptable salts. The term “physiologically acceptable salts” especially designates:
(1) pharmaceutically acceptable acid addition salts formed with pharmaceutically acceptable inorganic acids such as hydrochloric acid, hydrobromic acid, sulfuric acid, nitric acid, phosphoric acid and the like; or formed with pharmaceutically acceptable organic acids such as formic acid, acetic acid, benzenesulfonic acid, benzoic acid, camphorsulfonic acid, citric acid, ethane sulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like, and
(2) pharmaceutically acceptable base addition salts formed when an acidic proton present in the parent compound is either replaced by a metal ion, for example an alkali metal ion (e.g. Na, K), an alkaline earth metal ion (e.g. Ca, Mg), a zinc ion, a silver ion or an aluminum ion; or is coordinated with a pharmaceutically acceptable organic base such as diethanolamine, ethanolamine, N-methylglucamine, triethanolamine, tromethamine and the like; or with a pharmaceutically acceptable inorganic base such as aluminum hydroxide, calcium hydroxide, potassium hydroxide, sodium carbonate, sodium hydroxide and the like.

The “degree of modification” (MOD) of a polysaccharide, such as hyaluronic acid, is the molar quantity of cross-linking agent bound to the polysaccharide by one or more of its ends, expressed per 100 moles of repeating units of the polysaccharide. It can be determined by methods known to the person skilled in the art, such as nuclear magnetic resonance spectroscopy (NMR).

The “molar crosslinking rate” (CR), expressed in %, designates the molar ratio of the quantity of crosslinking agent relative to the quantity of repeating unit of the polysaccharide introduced into the crosslinking reaction medium expressed per 100 moles of repeating units of the polysaccharide in the crosslinking medium.

The expression “therapeutic active ingredient” designates a substance to cure or relieve symptoms and/or prevent a disease; a substance having curative or preventive properties with regard to human or animal diseases, as well as any substance which can be used in humans or animals or which can be administered to them, with a view to establishing a medical diagnosis or to restoring, correcting or modifying their physiological functions by exerting a pharmacological, immunological or metabolic action.

The expression “cosmetic active ingredient” designates any non-therapeutic substance especially intended to be brought into contact with various superficial parts of the human body, such as the epidermis, hair, nails, lips, chest and teeth, with a view, exclusively or principally, to cleaning, protecting, perfuming, maintaining in good condition or modifying their appearance or odor.

The term “approximately” means that the value concerned may be 10%, especially 5%, in particular 1%, lower or higher than the value indicated.

The expression “spacer group” designates a fragment comprising at least one atom intended to bond together two chemical groups within the same molecule. Preferentially, the spacer group contains at least one carbon atom.

The term “halogen” designates a fluorine, chlorine, bromine or iodine atom.

An “epoxide” group is an ethylene oxide residue bound to the rest of the molecule by one of its carbon atoms.

A “N-succinimidyloxycarbonyl” group is a group of chemical formula GR1 below:

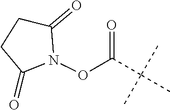

An “N-sulfosuccinimidyloxycarbonyl” group is a group of chemical formula GR2 below:

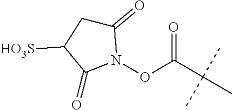

A “halocarbonyl” group is a group of formula —CO-Hal with Hal representing a halogen, such as Cl or Br.

A “carbodiimide” group is a group comprising an —N═C═N— unit, and more particularly a group of formula —N═C═N—R^{a} with R^{a} representing an aliphatic hydrocarbon group having from 1 to 20 carbon atoms, preferably a (C1-C6)alkyl group, of which one or more carbon atoms are optionally replaced by a heteroatom chosen from O, S and N, especially N.

An “acid anhydride residue” is a group comprising a —C(O)—O—C(O)— unit, and more particularly a monovalent cyclic group comprising the —C(O)—O—C(O)— unit, such as a monocyclic monovalent saturated hydrocarbon group comprising 5 to 10, especially 5 or 6, carbon atoms of which three successive carbon atoms are replaced by —C(O)—O—C(O)— and optionally one or more, especially one, additional carbon atoms, preferably not consecutive to the three carbon atoms substituted by —C(O)—O—C(O)—, are each replaced by a heteroatom such as N, O or S, especially N. The acid anhydride residue may correspond in particular to chemical formula GR3 below:

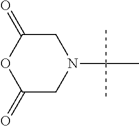

The acid anhydride residue may also be chosen from a maleic anhydride residue or a succinic anhydride residue.

The expression “aliphatic hydrocarbon chain” or “aliphatic hydrocarbon group” designates a linear, branched and/or cyclic, saturated or unsaturated but non-aromatic hydrocarbon group, advantageously comprising from 1 to 50, especially from 1 to 20, for example from 1 to 12 or from 1 to 6 carbon atoms. These will be alkyl groups in particular.

The expression “branched aliphatic hydrocarbon chain” specifically designates a main aliphatic hydrocarbon chain comprising at least one secondary aliphatic hydrocarbon chain.

The expression “star-shaped aliphatic hydrocarbon chain” designates a branched aliphatic hydrocarbon chain comprising several secondary aliphatic hydrocarbon chains all starting from a single branching point.

The expression “C1-Cx alkyl” or “(C1-Cx)alkyl” or alternatively “alkyl having from 1 to x carbon atoms” designates a saturated, linear or branched, monovalent hydrocarbon group having from 1 to x carbon atoms, with x being an integer, such as for example a methyl, ethyl, isopropyl, tert-butyl, n-pentyl, cyclopropyl, cyclohexyl, etc. group.

The expression “(C1-Cx)alkylene” designates a saturated, linear or branched, divalent hydrocarbon group having from 1 to x carbon atoms, with x being an integer, such as, for example, a methane-1,1-diyl, ethane-1, 1-diyl, ethane-1,2-diyl, propane-1,3-diyl, butane-1,4-diyl, butane-1,3-diyl, butane-1,2-diyl, pentane-1,5-diyl, hexane-1,6-diyl, hexane-1,5-diyl, heptane-1,7-diyl, octane-1,8-diyl, nonane-1,9-diyl, decane-1,10-diyl, etc. It is especially a methane-1,1-diyl or propane-1,3-diyl group.

The expression “hydroxy-(C1-Cx)alkyl” designates a (C1-Cx)alkyl group as defined above substituted by a hydroxyl (OH) group such as, for example, hydroxyethyl (—CH₂CH₂OH) or hydroxypropyl (for example —CH₂—CH(OH)—CH₃).

The expression “carboxy-(C1-Cx)alkyl” designates a (C1-Cx)alkyl group as defined above substituted by a carboxyl group (COOH) such as, for example, carboxymethyl (—CH₂COOOH).

The expression “aryl” designates a monovalent aromatic hydrocarbon group, preferably having from 6 to 10 carbon atoms, comprising one or more rings, such as, for example, a phenyl or naphthyl group.

The expression “arylene” designates a divalent aromatic hydrocarbon-based group, preferably having from 6 to 10 carbon atoms, comprising one or more rings, such as a phenylene group.

The expression “aryl-(C₁-Cx)alkyl” designates an aryl group as defined above, linked to the remainder of the molecule via a (C1-Cx)alkyl chain as defined above with x an integer, such as, for example, the benzyl or phenylethyl group.

The expression “polyvalent group” designates a group that can form several covalent bonds with other groups of the same compound or two different compounds. The bonds to the other groups may be formed from the same atom of the polyvalent group or from different atoms of the polyvalent group, and preferably from different atoms of the polyvalent group. In particular, the polyvalent group is a divalent group and can therefore form two covalent bonds with two other groups of the same compound or two different compounds. The number of covalent bonds that can be formed designates the valence of the polyvalent group.

### DETAILED DESCRIPTION OF THE INVENTION

The inventors have developed a method making it possible to prepare a hydrogel that satisfies the expressed needs. The hydrogel obtained has a stickiness allowing the hydrogel to adhere to the tissues and thus not to migrate in the tissues after its application/injection.

Currently available fillers create volume to fill soft tissues but do not combat tissue sagging. However, chronological aging of the skin is associated with a modification of the structure of the skin with the appearance of wrinkles but also ptosis of fatty areas. Interestingly, in certain embodiments, due to its extensibility, the hydrogel according to the invention can be used to retain tissues and prevent age-related tissue sagging, especially when it is administered and arranged in mesh form. The hydrogel according to the present invention is therefore most particularly useful in filling and/or replacing soft tissues.

Moreover, the hydrogel obtained is highly biocompatible. Indeed, its preparation method uses a very small amount of crosslinking agent (from 0.001 to less than 0.02 mole of crosslinking agent per 1 mole of polysaccharide repeating unit). The hydrogel obtained is thus weakly crosslinked.

In addition, the hydrogel obtained is highly mucoadhesive, which allows adhesion to the mucous membranes and thus good persistence of the hydrogel at the site(s) of administration. This is visible by qualitative ex vivo tests or in vitro tests that trace labeled components.

The method for preparing a hydrogel, preferably injectable, as developed by the inventors, comprises the following steps:
a) providing at least one polysaccharide;
b) providing at least one cross-linking agent, said cross-linking agent comprising at least two functional groups Z, identical or different, chosen from isocyanate, amino, epoxide, carboxyl, N-succinimidyloxycarbonyl, N-sulfosuccinimidyloxycarbonyl, halocarbonyl, isothiocyanate, vinyl, formyl, hydroxyl, sulfhydryl, hydrazino, acylhydrazino, aminoxy, carbodiimide, and an acid anhydride residue;
c) preparing a cross-linking reaction medium comprising the polysaccharide(s), the crosslinking agent(s) and a solvent, the total quantity of crosslinking agent ranging from 0.001 to less than 0.02 mole per 1 mole of polysaccharide repeating unit, the duration of the reaction medium preparation step not exceeding 5 hours;
d) placing the reaction medium obtained at the end of step c), for a period ranging from 2 weeks to 17 weeks, at a pressure P less than or equal to atmospheric pressure and at a temperature T greater than the eutectic point temperature of the reaction medium as measured at pressure P and less than the freezing point temperature of the reaction medium as measured at pressure P.

In the above method, the polysaccharide(s) and/or the cross-linking agent(s) may be in the form of a salt.

It will be readily understood that in the method of the present invention, the crosslinking of the polysaccharide mainly takes place during step d), i.e., when the reaction medium is in a frozen state.

The properties of the hydrogels obtained by the method of the present invention are most particularly surprising since, when it was a question of optimal crosslinking temperature, the prior art advised against going below 10° C. and remained silent on the use of temperatures below 0° C. (Jaeuk Baek, Yingfang Fan, Seol-Ha Jeong, Ho-Yong Lee, Hyun-do Jung, Hyoun-Ee Kim, Sukwha Kim, Tae-Sik Jang. Facile strategy involving low-temperature chemical cross-linking to enhance the physical and biological properties of hyaluronic acid hydrogel, Carbohydrate Polymers 202 (2018) 545-553).

Moreover, when it was a question of cryogelation, the quantities of cross-linking agent used remained very high (Anna Strom, Anette Larsson, Oguz Okay, Preparation and physical properties of hyaluronic acid-based cryogels, J. Appl. Polym. Sci. 2015, 42194; WO201029344). Otherwise, the prepared products were not able to withstand the heat sterilization conditions generally applied to this type of product (autoclave sterilization). These unstable to sterilization products ultimately have a predominant viscous component and/or a large fraction of polymers of low weight average molecular weight, which is not desirable for soft tissue filling applications. However, for obvious safety reasons, it is always desirable to propose products comprising as few chemical modifications as possible.

The hydrogels of the present invention differ from the hydrogels prepared with the same components but without freezing by their macroporous structure, i.e., with interconnected macropores, whose size varies from less than one micron to several hundred microns, depending on the synthesis conditions, that is, as a function of the nature and quantity of the reactants as well as a function of the temperature and the freezing rate. These macropores are visible during morphological analysis by scanning electron microscopy in the frozen state or by confocal laser scanning microscopy after staining the gel, for example with Rhodamine B.

Hydrogels which have been brought to room temperature after a crosslinking step in a frozen state may be designated by the term “cryogel”.

Moreover, although it was known that cryogelation generates only a few hyaluronic acid fragments, it remains surprising that the method of the present invention preserves the structure of polysaccharides of high weight average molecular weight, in particular the structure of hyaluronic acid of high weight average molecular weight.

The method according to the invention allows the preparation of a crosslinked polysaccharide gel without degradation into fragments of low weight average molecular weight.

A polysaccharide of high weight average molecular weight designates a polysaccharide whose weight average molecular weight corresponds to that of polysaccharides synthesized naturally (biologically). For example, a hyaluronic acid polysaccharide of high weight average molecular weight designates a polysaccharide which has a weight average molecular weight greater than or equal to 1 MDa, preferentially ranging from 1 MDa to 5 MDa, for example from 1 MDa to 3 MDa or from 1.5 MDa to 3 MDa; a chondroitin sulfate or chondroitin polysaccharide of high weight average molecular weight designates a polysaccharide greater than or equal to 30 kDa, preferentially ranging from 30 to 150 kDa, a high weight average molecular weight chitosan polysaccharide designates a polysaccharide which has an average molecular weight greater than or equal to 100 kDa, preferentially ranging from 100 kDa to 250 kDa. The polysaccharide supplied in step a) of the method is typically a polysaccharide of high weight average molecular weight.

A low weight average molecular weight polysaccharide designates a polysaccharide which has a significantly lower average molecular weight than those of biologically synthesized polysaccharides, more particularly a low weight average molecular weight polysaccharide designates a polysaccharide that has a weight average molecular weight less than or equal to one third of the weight of the high molecular weight polysaccharide supplied in step a) of preparing the hydrogel.

For example, a low weight average molecular weight hyaluronic acid polysaccharide designates a polysaccharide that has a weight average molecular weight ranging from 0.04 to 0.3 MDa, preferentially from 0.08 to 0.20 MDa, more preferentially from 0.08 to 0.15 MDa; a low weight average molecular weight chondroitin sulfate or chondroitin polysaccharide designates a polysaccharide that has a weight average molecular weight of less than or equal to 50 kDa; a low weight average molecular weight chitosan polysaccharide designates a polysaccharide that has a weight average molecular weight of less than or equal to 30 kDa. The absence of low molecular weight fragments is advantageous. Indeed, although hyaluronic acid is known to be biocompatible, it has already been reported in the literature that hyaluronic acid of low weight average molecular weight may cause long-term adverse effects after injection into a patient (Cyphert J M, Trempus C S, Garantziotis S. Size Matters: Molecular Weight Specificity of Hyaluronan Effects in Cell Biology. International Journal of Cell Biology 2015; 2015:563818).

### Components Used in the Preparation of the Hydrogel

### Polysaccharides

The polysaccharide can be any polymer composed of monosaccharides joined together by glycosidic bonds.

Preferably, the polysaccharide is chosen from pectin and pectic substances; chitosan; chitin; cellulose and its derivatives; agarose; glycosaminoglycans such as hyaluronic acid, heparosan, dermatan sulfate, keratan sulfate, chondroitin and chondroitin sulfate; and their mixtures.

Pectic substances, including pectin, are polysaccharides composed of a skeleton of D-galacturonic acid in acid form possibly esterified with methanol and L-rhamnose capable of branching with other monosaccharides.

Chitosan and chitin are each a polysaccharide composed of D-glucosamine repeating units linked to each other by a B-(1,4) bond, part of which is N-acetylated. More particularly, chitosan has a degree of acetylation of less than 50% while chitin has a degree of acetylation of more than 50%.

Cellulose is a polysaccharide composed of a linear chain of D-glucose molecules.

Cellulose derivatives include methylcellulose, ethylcellulose, ethylmethylcellulose, hydroxypropylmethylcellulose (HPMC), hydroxyethylcellulose (HEC), hydroxypropylcellulose (HPC) and carboxymethylcellulose (CMC).

Agarose is a polysaccharide comprising as a repeating unit a disaccharide of D-galactose and 3,6-anhydro-L-galactopyranose.

Glycosaminoglycans are linear polysaccharides composed of repeating units of disaccharides, the disaccharides containing a hexosamine (glucosamine (GlcN) or galactosamine (GalN)) and another monosaccharide (glucuronic acid (GlcA), iduronic acid (IdoA) or galactose (Gal)). The hexosamine and the other monosaccharide can optionally be sulfated and/or acetylated. The glycosaminoglycan can especially be hyaluronic acid, heparosan, dermatan sulfate, keratan sulfate, chondroitin or chondroitin sulfate.

Hyaluronic acid is a glycosaminoglycan whose repeating unit is a disaccharide composed of D-glucuronic acid and N-acetyl-D-glucosamine, linked together by alternating glycosidic β-(1,4) and β-(1,3) bonds. When hyaluronic acid is in the form of a salt, it is also referred to as hyaluronate or hyaluronan.

In the context of the present invention, hyaluronic acid may have a weight average molecular weight ranging from 0.05 to 10 MDa, preferably ranging from 0.5 to 5 MDa, even more preferentially greater than 0.05 MDa, for example ranging from 0.07 to 10 MDa or from 0.07 to 5 MDa, or from 0.5 to 5 MDa or from 1 to 5 MDa or from 2 to 4 MDa. Hyaluronic acid can be in salt form, in particular in physiologically acceptable salt form such as sodium salt, potassium salt, zinc salt, calcium salt, magnesium salt, silver salt, calcium salt and mixtures thereof. More particularly, hyaluronic acid is in acid form or in sodium salt form (NaHA).

Heparosan is a glycosaminoglycan whose repeating unit is a disaccharide composed of glucuronic acid (GlcA) linked by an a-(1,4) bond to an N-acetyl glucosamine (GlcNAc). Each disaccharide repeating unit is connected to the next by a β-(1,4) bond.

Chondroitin sulfate is a glycosaminoglycan whose repeating unit is a disaccharide composed of glucuronic acid linked by a β-(1,3) bond to sulfated N-acetyl galactosamine, i.e., it comprises at least one sulfate substituent. Each disaccharide repeating unit is connected to the next by a β-(1,4) bond.

Dermatan sulfate is a glycosaminoglycan whose repeating unit is a sulfated disaccharide, i.e., comprising at least one sulfate substituent, of L-iduronic acid and of N-acetyl-galactosamine-linked by an α-(1,3) bond. Advantageously, the disaccharide is sulfated at the C-4 position of N-acetylgalactosamine, at the C-6 position of N-acetylgalactosamine, at the C-2 position of L-iduronic acid, or at a combination of these positions. Each disaccharide repeating unit is connected to the next by a s-(1,4) bond.

Keratan sulfate is a glycosaminoglycan whose repeating unit is a sulfated disaccharide, i.e., comprising at least one sulfate substituent, composed of D-galactose and N-acetylglucosamine linked by alternating β-(1,4) and β-(1,3) bonds.

The polysaccharide may be in the form of a salt, in particular in the form of a physiologically acceptable salt such as sodium salt, potassium salt, zinc salt, calcium salt, magnesium salt, silver salt and mixtures thereof, more particularly in the form of a sodium or potassium salt.

Advantageously, the polysaccharide is a glycosaminoglycan or a salt thereof, preferentially hyaluronic acid or a salt thereof, more preferentially hyaluronic acid or one of its physiologically acceptable salts such as sodium salt, potassium salt, zinc salt, silver salt and mixtures thereof, even more preferentially hyaluronic acid or its sodium salt.

The polysaccharide generally has a weight average molecular weight ranging from 0.03 to 10 MDa.

Preferably, if the polysaccharide is hyaluronic acid, it has weight average molecular weight (MW) ranging from 0.05 to 10 MDa, preferentially ranging from 0.5 to 5 MDa, even more preferentially greater than 0.05 MDa, for example ranging from 0.07 to 10 MDa or from 0.07 to 5 MDa, or from 0.5 to 5 MDa or from 1 to 5 MDa or from 2 to 4 MDa.

### Crosslinking Agent

The crosslinking agent is a compound comprising at least two functional groups capable of covalently bonding with functional groups present on the polysaccharide, such as OH, CHO, NH₂ or COOH groups carried by the polysaccharide, and thus of inducing bonds between polysaccharide chains (cross-linking) and/or bonds on the same polysaccharide chain.

The cross-linking agent useful in the context of the present invention comprises at least two, preferably from 2 to 8, especially 2, functional groups (designated “group Z”) preferably chosen independently from isocyanate (—N═C═O), amino (—NH₂), epoxide, carboxyl (—COOH), N-succinimidyloxycarbonyl, N-sulfosuccinimidyloxycarbonyl, halocarbonyl, isothiocyanate (—N═C═S), vinyl (—CH═CH₂), formyl (—CH═O), hydroxyl (—OH), sulfhydryl (—SH), hydrazino (—NH—NH₂), acylhydrazino (—CO—NH—NH₂), aminoxy (—O—NH₂), carbodiimide groups, and an acid anhydride residue. Preferably, the functional groups are identical.

The isocyanate group can react with an OH or NH₂ group of the polysaccharide to form a carbamate or urea function. The amino group can react with a COOH group of the polysaccharide to form an amide function. The epoxide group can react with an OH or COOH group of the polysaccharide to form an ether or ester function. The carboxyl group can react with an OH or NH₂ group of the polysaccharide to form an ester or amide function. The N-succinimidyl oxycarbonyl and N-sulfosuccinimidyl oxycarbonyl groups can react with an OH or NH₂ group of the polysaccharide to form an ester or amide function. The halocarbonyl group can react with an OH or NH₂ group of the polysaccharide to form an ester or amide function. The isothiocyanate group can react with an OH or NH₂ group of the polysaccharide to form a thiocarbamate or thiourea function. The vinyl group can react with an OH group of the polysaccharide to form an ether function. The formyl group can react with an OH or NH₂ group of the polysaccharide to form hemiacetal or hemiaminal function. The hydroxyl group can react with a COOH group of the polysaccharide to form an ester function. The sulfhydryl group can react with a COOH group of the polysaccharide to form a thioester function. The hydrazino (—NH—NH₂) group can react with an CHO group of the polysaccharide to form a hydrazone function. The acylhydrazino group can react with a CHO group of the polysaccharide to form a carbonylated hydrazone ═NNHC(O)— function. The aminoxy group can react with a CHO group of the polysaccharide to form an oxime ═NO— function. The carbodiimide group can react with a COOH group of the polysaccharide to give a CO—NR^{a}—CO—NH function, and an acid anhydride residue can react with an OH or NH₂ group of the polysaccharide to form an ester or amide function.

Preferably, the functional groups Z are identical and represent an epoxide or vinyl group, more preferentially an epoxide group.

According to another advantageous embodiment, the functional groups Z are identical and chosen from amino, vinyl, formyl and carbodiimide groups, preferably amino groups.

In particular, the cross-linking agent is chosen from hexamethylene diisocyanate, diphenylmethylene 4,4′-diisocyanate, 4arm PEG20K-isocyanate, spermine (or 1,12-diamino-5,9-diazadodecane), spermidine (or 1,8-diamino-5-azaoctane), cadaverine (or 1,5-diaminopentane), putrescine (or 1,4-diaminobutane), poly(ethylene glycol) diamine, ethylenediamine, 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end with a diglycidyl ether (CAS number: 130167-23-6), poly(ethylene glycol) diacid, disuccinimidyl suberate, bis(sulfosuccinimidyl)suberate, sebacoyl chloride, 1,4-butane diisothiocyanate, divinylsulfone (DVS), glutaraldehyde, polyethylene glycol, 1,5-pentanedithiol, adipic acid dihydrazide, bis-aminooxy-poly(ethylene glycol), diethylenetriaminepentaacetic acid dianhydride, and their mixtures.

When the functional groups Z are epoxide groups, the cross-linking agent is preferably chosen from 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end by a diglycidyl ether (CAS number: 130167-23-6), hydroxyapatite beads modified to carry epoxy groups, and their mixtures.

More preferentially, the cross-linking agent is chosen from 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), and their mixtures.

When the functional groups Z are amino groups, the cross-linking agent is preferably a polyamine chosen from spermine (or 1,12-diamino-5,9-diazadodecane), spermidine (or 1,8-diamino-5-azaoctane), cadaverine (or 1,5-diaminopentane), putrescine (or 1,4-diaminobutane), their salts or a mixture thereof, more preferentially the crosslinking agent is a polyamine chosen from spermine, spermidine and their mixtures.

The crosslinking agent may be chosen from hydroxyapatite beads modified to carry epoxy groups, a compound of chemical formula I as described below, and their mixtures.

Preferably, the cross-linking agent is a compound of chemical formula I: Y—(Z)ₙ
in which the functional groups Z, identical or different, are as defined above, n is an integer greater than or equal to 2, especially ranging from 2 to 8, preferably equal to 2, Y is a polyvalent hydrocarbon group, especially aliphatic, having a valence of n and having from 1 to 150 carbon atoms:
in which one or more (for example 1 to 150, or else 1 to 50 or else 1 to 15 or else 1 or 2) CH₂ units are optionally replaced by one or more divalent units chosen from arylenes; —O—; —S—; —S(O)—; —C(═O)—; —SO₂—; —N(R¹)—; and —[SiR²R³O]m-SiR²R³— with:
   R¹ representing a hydrogen atom, an aliphatic hydrocarbon group having from 1 to 6 carbon atoms, or an aryl-(C1-C6)alkyl;
   m an integer comprised between 1 and 20 and
   R² and R³, identical or different, representing a hydrogen atom; a halogen atom; an —OR″ group with R″ representing a hydrogen atom, an aryl group or an aliphatic hydrocarbon group having from 1 to 6 carbon atoms; an aryl; or an aliphatic hydrocarbon group having from 1 to 6 carbon atoms optionally substituted by one or more groups chosen from a halogen atom, an aryl or a hydroxyl,
said polyvalent group being unsubstituted or substituted by one or more monovalent groups chosen from a halogen atom, a hydroxyl, and an aryl-(C1-C6)alkyl, preferably unsubstituted.

In particular, n is an integer ranging from 2 to 8, preferably n represents 2, 3 or 4, even more preferentially n is equal to 2.

Advantageously, R¹ represents a hydrogen atom or a (C1-C6)alkyl group.

In particular, R² and R³, identical or different, represent an aliphatic hydrocarbon group having from 1 to 6 carbon atoms, more particularly a (C1-C6)alkyl group.

Preferably, in the definition of Y, the polyvalent hydrocarbon group may be an aliphatic or aromatic polyvalent hydrocarbon group, preferably aliphatic and especially saturated, with a valence of n and having from 1 to 150 carbon atoms, preferentially from 1 to 50 carbon atoms, more preferentially from 1 to 20 carbon atoms, even more preferentially from 2 to 20 carbon atoms.

In particular, in the definition of Y, the polyvalent hydrocarbon group is a saturated, especially linear, aliphatic polyvalent hydrocarbon group.

Preferably, Y is a polyvalent hydrocarbon group as described above in which one or more CH₂ units are optionally replaced by one or more divalent units chosen from —O—, —SO₂—, —[SiR²R³O]m-SiR²R³— and —NH—, with R², R³ and m as described above.

In particular, Y is a polyvalent hydrocarbon group as described above, preferably aliphatic and saturated, and especially linear, branched or star-shaped, and optionally in which:
at least two CH₂ units are replaced by —O—, particularly between 1 and 50 CH₂ units, more particularly between 1 and 15 CH₂ units, or
at least one, preferably one or two, CH₂ units is replaced by an —NH— unit, or
at least one, preferably one, CH₂ unit is replaced by an —SO₂— unit, or
at least two, preferably two, CH₂ units are replaced by —O— and at least one, preferably one, CH₂ unit is replaced by an —[SiR²R³O]m-SiR²R³— unit with R², R³ and m as described above.

More particularly, when one or more CH₂ units are replaced by —O—, the unit or units replaced are such that Y comprises one or more —CH₂—CH₂—O— units. In particular, Y comprises from 1 to 50 —CH₂—CH₂—O— units, advantageously from 2 to 25 —CH₂—CH₂—O— units, more advantageously from 2 to 15 —CH₂—CH₂—O— units. Y may comprise only —CH₂—CH₂—O— units.

More preferentially, Y is an alkyl group comprising 1 to 150, especially 1 to 50, in particular 1 to 20, for example 1 to 12, especially 1 to 6 carbon atoms, preferably linear, in which optionally one or more CH₂ units are replaced by one or more divalent units chosen from —O— and —NH—, more particularly between 1 and 50, especially between 1 and 15, for example 1 or 2, divalent units chosen from —O— and —NH—.

According to a first embodiment, R² and R³, identical or different, represent an —OR″ group with R″ as described above. In particular, R″ represents an aliphatic hydrocarbon group having from 1 to 6 carbon atoms, more particularly a (C1-C6)alkyl group.

According to a second embodiment, R² and R³, which may be identical or different, represent an aliphatic hydrocarbon group having from 1 to 6 carbon atoms optionally substituted (preferably unsubstituted) by one or more groups chosen from a halogen atom, an aryl or a hydroxyl, more preferentially an unsubstituted (C1-C6)alkyl group such as a methyl or an ethyl.

Advantageously, the cross-linking agent is a compound of chemical formula Ia below: Z¹—Y¹—Z²
in which identical or different groups Z¹ and Z² are chosen from isocyanate, amino, epoxide, carboxyl, N-succinimidyloxycarbonyl,
N-sulfosuccinimidyloxycarbonyl, halocarbonyl, isothiocyanate, vinyl, formyl, hydroxyl, sulfhydryl, hydrazino, acylhydrazino, aminoxy, carbodiimide, and an acid anhydride residue, and Y¹ represents a hydrocarbon divalent chain, especially aliphatic, having from 1 to 50 carbon atoms:
in which one or more (e.g.: 1 to 15 or else 1 or 2) CH₂ units are optionally replaced by one or more divalent units chosen from arylenes, —O—, —S—, —S(O)—, —C(═O)—, —SO₂—, —N(R¹)—, and —[SiR²R³O]m-SiR²R³— with
R¹ representing a hydrogen atom, an aliphatic hydrocarbon group having from 1 to 6 carbon atoms, or an aryl-(C1-C6)alkyl,
m an integer comprised between 2 and 20, and
R² and R³, identical or different, representing a hydrogen atom; a halogen atom; an —OR″ group with R″ representing a hydrogen atom, an aryl group or an aliphatic hydrocarbon group having from 1 to 6 carbon atoms; an aryl; or an aliphatic hydrocarbon group having from 1 to 6 carbon atoms optionally substituted by one or more groups chosen from a halogen atom, an aryl or a hydroxyl,
said chain being unsubstituted or substituted by one or more monovalent groups chosen from a halogen atom, a hydroxyl or a aryl(C1-C6)alkyl.

The groups Z¹ and Z² have the same definition as the group Z defined above.

Y¹ has the same definition as Y defined above with a valence n being equal to 2.

In particular, Y¹ may comprise only —CH₂—CH₂—O— units, as defined above.

Preferably, the cross-linking agent of chemical formula I or Ia does not include —[SiR²R³O]ₘSiR²R³— units.

### Other Components

Additional components can be lubricating agents; cosmetic active ingredients such as antioxidants, co-enzymes, amino acids, vitamins, minerals, and nucleic acids; therapeutic active ingredients such as anesthetics, antibiotics, antifungals, and adrenaline and its derivatives, and mixtures thereof.

Polysaccharides, in particular non-crosslinked hyaluronic acid or non-crosslinked heparosan, can be mentioned as examples of lubricating agents.

Examples of anesthetics include, in a non-limiting manner, ambucaine, amoxecaine, amylocaine, aprindine, aptocaine, articaine, benzocaine, betoxycaine, bupivacaine, butacaine, butamben, butanilicaine, chlorobutanol, chloroprocaine, cinchocaine, clodacaine, cocaine, cryofluorane, cyclomethycaine, dexivacaine, diamocaine, diperodon, dyclonine, etidocaine, euprocin, febuverine, fomocaine, guafecainol, heptacaine, hexylcaine, hydroxyprocaine, hydroxytetracaine, isobutamben, leucinocaine, levobupivacaine, levoxadrol, lidamidine, lidocaine, lotucaine, menglytate, mepivacaine, meprylcaine, myrtecaine, octacaine, octodrine, oxetacaine, oxybuprocaine, parethoxycaine, paridocaine, phenacaine, piperocaine, piridocaine, polidocanol, pramocaine, prilocaine, procaine, propanocaine, propipocaine, propoxycaine, proxymetacaine, pyrrocaine, quatacaine, quinisocaine, risocaine, rodocaine, ropivacaine, tetracaine, tolycaine, trimecaine, a salt of their salts, in particular a hydrochloride, or a mixture thereof.

Examples of antioxidants include, in a non-limiting manner, glutathione, reduced glutathione, ellagic acid, spermine, resveratrol, retinol, L-carnitine, polyols, polyphenols, flavanols, theaflavins, catechins, caffeine, ubiquinol, ubiquinone, alpha-lipoic acid and their derivatives, and a mixture thereof.

Examples of amino acids include, in a non-limiting manner, arginine (e.g. L-arginine), isoleucine (e.g. L-isoleucine), leucine (e.g. L-leucine), lysine (e.g. L-Lysine or L-Lysine monohydrate), glycine, valine (e.g. L-valine), threonine (e.g. L-threonine), proline (e.g. L-proline), methionine, histidine, phenylalanine, tryptophan, cysteine, their derivatives (e.g. N-acetylated derivatives such as N-acetyl-L-cysteine) and a mixture thereof.

Examples of vitamins and their salts include, in a non-limiting manner, vitamins E, A, C, B, especially vitamins B6, B8, B4, B5, B9, B7, B12, and better still pyridoxine and its derivatives and/or salts, preferably pyridoxine hydrochloride.

Examples of minerals include, in a non-limiting manner zinc salts (e.g. zinc acetate, especially dehydrated), magnesium salts, calcium salts (e.g. hydroxyapatite, especially in bead form), potassium salts, manganese salts, sodium salts, copper salts (e.g. copper sulfate, especially pentahydrate), optionally in hydrated form, and mixtures thereof.

Examples of nucleic acids include, in a non-limiting manner, adenosine, cytidine, guanosine, thymidine, cytosine, their derivatives and a mixture thereof.

Co-enzymes include coenzyme Q10, CoA, NAD, NADP, and mixtures thereof.

Adrenaline derivatives include adrenaline, noradrenaline and a mixture thereof.

The quantities of additional compounds obviously depend on the nature of the compound in question, on the desired effect, and on the destination of the composition as described here.

### Method

The steps of the method of the present invention can be as described below.

### Providing at Least One Polysaccharide or Salt Thereof (Step a))

Step a) of the method according to the invention comprises providing at least one polysaccharide or a salt thereof, in particular a physiologically acceptable salt thereof.

The polysaccharide is as described above. Preferably, the polysaccharide is hyaluronic acid or a salt of hyaluronic acid, preferably a sodium salt.

The polysaccharide may be provided in hydrated form, totally or partially, or in dry form, such as in powder or fiber form. More particularly, in step a), the polysaccharide is provided in dry form such as in powder or fiber form.

When the polysaccharide is supplied in hydrated form, it is in the form of a non-crosslinked gel or solution.

In particular, when the polysaccharide is in hydrated form, it is an aqueous non-crosslinked gel or aqueous solution. More particularly, the polysaccharide is mixed with water, with the optional addition of a phosphate buffer or a supplemented phosphate buffer, i.e., possibly comprising additional components as defined in the additional steps. It should therefore be understood that the aqueous non-crosslinked gel or aqueous polysaccharide solution does not comprise sodium hydroxide.

### Providing at Least One Cross-Linking Agent or Salt Thereof (Step b)

Step b) of the method according to the invention comprises providing at least one cross-linking agent or a salt thereof, in particular a physiologically acceptable salt thereof.

The cross-linking agent is as described above.

### Preparing the Crosslinking Reaction Medium (Step c)

Step c) of the method according to the invention comprises preparing a crosslinking reaction medium. The reaction medium comprises the polysaccharide(s), the crosslinking agent(s) and a solvent.

The solvent is typically water or a mixture comprising water and an organic solvent (typically a mixture comprising at least 90% by weight of water, or at least 95% or at least 99% by weight of water relative to the total weight of the solvent). For example, an organic solvent such as an alcohol, particularly ethanol, or DMSO, can be used to solubilize the cross-linking agent, for example when it is poly(dimethylsiloxane) terminated at each end with a diglycidyl ether (CAS number: 130167-23-6), before its addition to the aqueous reaction medium.

The reaction medium may also comprise salts, pH adjusters, for example a Bronsted base, more preferentially a hydroxide salt, such as sodium or potassium hydroxide, additional components as described above and their mixtures. The addition of a Bronsted base can especially be necessary when the functional groups Z of the cross-linking agent, such as Z¹ or Z², represent an epoxide group or a vinyl group. In these cases, cross-linking takes place at a pH greater than or equal to 10, more advantageously greater than or equal to 12, which requires the addition of a Bronsted base to the reaction medium (for example sodium hydroxide), typically at a concentration comprised between 0.10 M and 0.30 M.

The reaction medium is typically prepared from the polysaccharide or polysaccharides in dry form. When the reaction medium is prepared from the polysaccharide or polysaccharides in hydrated form, the aqueous non-crosslinked gel or aqueous polysaccharide solution used for the preparation of the reaction medium does not comprise sodium hydroxide. Thus, the maximum contact time of the polysaccharide with sodium hydroxide before the initiation of step d), whether the polysaccharide is supplied in dry or hydrated form, is 5 hours, for example from 15 minutes to 4 hours or from 30 minutes to 2 hours.

The total quantity of crosslinking agent in the reaction medium varies from 0.001 to less than 0.02 moles per 1 mole of repeating unit of the polysaccharide, for example from 0.001 to 0.015 moles per 1 mole of repeating unit of the polysaccharide, preferably from 0.001 to 0.01 moles per 1 mole of repeating unit of the polysaccharide, even more preferably from 0.001 to 0.008 moles or from 0.001 to 0.005 moles per 1 mole of repeating unit of the polysaccharide. When the polysaccharide is a glycosaminoglycan such as hyaluronic acid, the repeating unit is a disaccharide unit.

The concentration by mass of polysaccharide or polysaccharide salt in the reaction medium advantageously ranges from 50 to 300 mg/g of solvent, preferably from 100 to 200 mg/g.

Step c) of the method according to the invention typically comprises a step of homogenizing the reaction medium. Homogenization is usually carried out by three-dimensional stirring, stirring with a mixer, stirring with blades or stirring with a spatula.

Step c) is typically carried out at a temperature ranging from 4 to 35° C., preferably 15° C. to 25° C.

The duration of the step of preparing the reaction medium does not exceed 5 hours. It generally varies from 15 minutes to 4 hours, preferably from 30 minutes to 2 hours. The reaction medium obtained at the end of step c) is advantageously placed directly under the conditions of step d) according to the invention.

### Crosslinking at Temperature T and Pressure P (Step d)

The reaction medium obtained at the end of step c) is then placed, for a period ranging from 2 weeks to 17 weeks, at a pressure P less than or equal to atmospheric pressure and at a temperature T greater than the eutectic point temperature of the reaction medium (i.e., of the mixture comprising the polysaccharide(s), the cross-linking agent(s), the solvent and optional salts, pH adjusters and additional components) as measured at the pressure P and below the freezing point temperature of the reaction medium as measured at the pressure P.

The freezing point temperature of the reaction medium designates the temperature at which the mixture of the components of the reaction medium, on a macroscopic scale, solidifies, i.e., becomes non-fluid. Below the freezing point, the mixture is in a freezing state which is characterized by the coexistence of components in solid and liquid form. The freezing state is maintained up to the eutectic point temperature of the reaction medium.

The eutectic point temperature of the reaction medium refers to the temperature below which the mixture of the components of the reaction medium changes from a freezing state (coexistence of liquid and solid phases) to a completely solid state, i.e., a state in which all the components of the mixture are in solid form.

The freezing point and eutectic point of a mixture depend on the pressure to which the mixture is subjected so the freezing point and eutectic point are measured at the pressure P.

The freezing point and eutectic point can be determined by differential scanning calorimetry.

This method makes it possible to determine the phase transitions.

To do this, the product to be studied is gradually cooled until it observes its phase transitions.

The temperature T is preferably greater than or equal to −55° C. and less than or equal to −5° C., preferably it ranges from −35° C. to −10° C. More preferably still, the temperature T is approximately −20° C.

The pressure P is preferably atmospheric pressure. Atmospheric pressure is the pressure exerted by the air making up the atmosphere on any surface in contact with it. It varies according to altitude. At an altitude of 0 m, the average atmospheric pressure is 101,325 Pa.

Preferably, the pressure P is atmospheric pressure, and the temperature T is greater than or equal to −55° C. and less than or equal to −5° C.; preferably T varies from −35° C. to −10° C. or is approximately −20° C.

Preferably, the reaction medium obtained at the end of step c) is placed for a period ranging from 2 weeks to 15 weeks, preferably from 2 weeks to 13 weeks, preferably from 2 weeks to 11 weeks, preferably from 2 weeks to 9 weeks, more preferably from 3 weeks to 6 weeks or else from approximately 4 to 5 weeks or approximately 30 days at said pressure P and at said temperature T.

As indicated above, the cross-linking of the polysaccharide mainly takes place during step d) (it can nevertheless start from step c). This step therefore makes it possible to cross-link the polysaccharide chains with one another. The functional groups of the crosslinking agent react with functional groups present on the polysaccharides to bind the polysaccharide chains with one another and to cross-link them by forming intermolecular bonds. The crosslinking agent can also react with functional groups present on the same polysaccharide molecule so as to form intramolecular bonds. In particular, the functional groups of the cross-linking agent react with the —OH or —COOH or —CHO groups present on polysaccharides such as hyaluronic acid. Crosslinked polysaccharides comprising at least one cross-linking link between two polysaccharide chains, said cross-linking link being the residue of the cross-linking agent, are thus obtained. In particular, following step d), the crosslinked polysaccharides comprise at least one cross-linking bond between two polysaccharide chains, said cross-linking bond comprising more particularly the polyvalent group Y as described above, preferably the divalent group Y¹ as described above. Certain functional groups Z (such as Z¹ and Z²) of the cross-linking agent may not react with a polysaccharide chain. In particular, when the cross-linking agent has two functional groups Z¹ and Z², one of the functional groups Z¹ may react with a polysaccharide while the other functional group Z² does not react with any polysaccharide. A pendant link is then formed.

The cross-linking can be carried out in the presence of several cross-linking agents. When cross-linking is carried out in the presence of several cross-linking agents, the cross-linking agents can be added simultaneously or separately over time to the reaction medium. Step b) may thus comprise repeated cross-linking steps. The cross-linking is then carried out in the presence of a total quantity of cross-linking agents ranging from 0.1 to less than 2 moles, preferably ranging from 0.1 to 1.5 moles or from 0.1 to 1 mole or from 0.1 to 0.8 moles or from 0.1 to 0.5 moles of cross-linking agents (or their salts) per 100 mole of repeating unit of the polysaccharide. The cross-linking conditions, in particular the contents of cross-linking agent, duration and temperatures, as well as the weight average molecular weights (Mw) of the polysaccharide used are interdependent.

More particularly, the higher the temperature of the cross-linking reaction, the shorter the reaction time can be to obtain the same degree of modification of the polysaccharide by the cross-linking agent.

The lower the content of cross-linking agent, the longer the reaction time must be in order to obtain similar mechanical properties of the resulting gel. In other words, the lower the molar percentage of cross-linking agent, the fewer reactive functional groups there are in the reaction medium and the lower the probability that 2 groups will meet and react together, the longer the reaction time must be in order to allow the functional groups to react with one another and form cross-linking bonds, and thus obtain a gel with desirable properties.

Conversely, too large a quantity of cross-linking agent and too long a cross-linking time will result in a gel that is brittle and too hard, and therefore not interesting for a cosmetic application.

The higher the weight average molecular weight (Mw) of the polysaccharide, the lower the degree of modification required to obtain gels with given mechanical properties.

For the same molar percentage of cross-linking agent, the lower the weight average molecular weight (Mw) of the polysaccharide, the longer the reaction time to be able to obtain gels with given mechanical properties.

When the functional groups Z of the cross-linking agent are amino groups, the cross-linking reaction with the polysaccharide is advantageously carried out in the presence of at least one activator, and if appropriate combined with at least one coupling aid.

In this respect, the activator can be chosen from water-soluble carbodiimides such as 1-ethyl-3-(3-dimethylaminopropyl)carbodiimide (EDC), 1-ethyl-3-[3-(trimethylamino)propyl]carbodiimide hydrochloride (ETC), 1-cyclohexyl-3-(2-morphilinoethyl)carbodiimide (CMC), their salts and mixtures thereof, preferably represented by EDC.

As for the coupling aid, when present, it may be chosen from N-hydroxy succinimide (NHS), N-hydroxybenzotriazole (HOBt), 3,4-dihydro-3-hydroxy-4-oxo-1,2,3-benzotriazole (HOOBt), 1-hydroxy-7-azabenzotriazole (HAt) and N-hydroxysulfosuccinimide (sulfo NHS), and mixtures thereof, preferably represented by HOBt.

### Additional Steps

The method according to the invention may comprise one or more additional steps, such as, for example, steps of addition of one or more additional components, purification, sterilization, sieving, swelling and/or packaging.

The method according to the invention may comprise a step of adding at least one additional component. The additional component can be chosen from lubricating agents; cosmetic active ingredients such as antioxidants, co-enzymes, amino acids, vitamins, minerals and nucleic acids; therapeutic active ingredients such as anesthetics, antibiotics, antifungals and adrenaline and its derivatives, and mixtures thereof. The additional components may be as described above.

The method according to the invention may comprise at least one purification step. Purification can be carried out by dialysis.

The method according to the invention may comprise a step of sterilizing the hydrogel. Sterilization is preferably carried out by heat. Sterilization is generally carried out by increasing the temperature of the sterilization medium to a temperature called the plateau temperature, which is maintained for a specified period called the plateau time. Sterilization is preferably carried out at a plateau temperature ranging from 121° C. to 135° C., preferably for a plateau time ranging from 1 minute to 20 minutes with F0≤15. The sterilizing value F0 corresponds to the time required, in minutes, at 121° C., to inactivate 90% of the population of microorganisms present in the product to be sterilized. Alternatively, sterilization can be carried out by gamma radiation, UV radiation or by means of ethylene oxide.

The method may comprise a step of sieving the hydrogel, more particularly with a sieve with a porosity comprised between 50 and 2000 μm. This sieving step makes it possible to obtain a more homogeneous hydrogel with an extrusion force that is as constant as possible, i.e. as regular as possible. The person skilled in the art knows how to select a sieve with a pore size adapted to the mechanical properties of the hydrogel being prepared.

The method may comprise a step of swelling the hydrogel. During the hydrogel swelling step, the polysaccharide concentration of the hydrogel is adjusted. In particular, a solvent is added, for example, water, phosphate buffer or water for injections. More particularly, the solvent added has a pH around physiological pH (6.8-7.8). The polysaccharide concentration obtained following the swelling step advantageously varies from 1 mg/g of gel to 50 mg/g of hydrogel, more advantageously from 5 mg/g to 35 mg/g of hydrogel, even more advantageously from 10 mg/g to 30 mg/g of hydrogel.

The method may comprise a step of packaging the hydrogel.

As applicable, the step of adding one or more additional components preferentially takes place after the purification step.

As applicable, the step of adding one or more additional components preferentially takes place before the sterilization step.

In particular, the step of adding one or more additional components may also comprise the addition of at least one therapeutic active ingredient, or at least one cosmetic active ingredient, or a mixture thereof. When at least one therapeutic active ingredient and/or at least one cosmetic active ingredient is added, the step of adding one or more additional components preferably takes place after step d).

As applicable, the purification step preferentially takes place after the step of adding one or more additional components.

As applicable, the purification step preferentially takes place before the sterilization step.

As applicable, the purification step preferentially takes place before the sieving step.

The sterilization step is preferably carried out after steps a) to d) and any additional steps. In particular, the hydrogel is sterilized after it has been packaged in its injection device and the gel is packaged after all the steps of the process and before sterilization.

### Hydrogel

Another object of the present invention is a hydrogel which can be obtained by the method of the present invention. Such a hydrogel can also be designated by the term cryogel.

The hydrogel comprises one or more polysaccharides as defined above. The polysaccharide is crosslinked. The molar degree of cross-linking of the polysaccharide is greater than 0 and less than 2%, preferably less than or equal to 1.5% or less than or equal to 1%, even more preferentially less than or equal to 0.8% or 0.5%, especially between 0.1% and 0.8% or between 0.1 and 0.5% (number of moles of cross-linking agent(s) per 100 moles of repeating unit of the polysaccharide(s)).

The cross-linking agent or agents are as defined above.

The cross-linking agent is preferably a cross-linking agent whose functional groups are epoxide groups. Thus, the cross-linking agent is preferably 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), and their mixtures.

The hydrogel of the present invention has a sticky character. In some embodiments, the hydrogel of the present invention has an extensible character.

The hydrogel is preferably an injectable hydrogel.

It is preferably sterile, especially heat sterilized at a plateau temperature of 121° C. to 135° C., preferably for a plateau time ranging from 1 minute to 20 minutes with F0≤15.

This hydrogel is preferably homogeneous.

The hydrogel may comprise one or more additional components as described above, in particular one or more additional components chosen from lubricants, anesthetics, antioxidants, amino acids, vitamins, minerals, nucleic acids and their mixtures.

Preferentially, a hydrogel according to the present invention, acceptable for the therapeutic and/or cosmetic applications targeted by the present invention, has a cross-over stress (or stress at the intersection of the G′ and G″ moduli) greater than or equal to 50 Pa, preferentially between 50 and 5000 Pa and even more preferentially between 100 and 1000 Pa, preferably greater than 150 Pa and an elastic modulus G′ greater than or equal to 20 Pa, preferentially from 100 Pa to 2000 Pa, still more preferentially from 110 Pa to 1000 Pa.

Preferentially, a hydrogel according to the present invention, acceptable for the therapeutic and/or cosmetic applications targeted by the present invention, has a cohesivity of 1 N to 30 N, preferably greater than 4 N, for example ranging from 6 to 20 N or ranging from 6 to 15 N. This cohesivity is measured by mechanical compression using a rheometer. For this, the gel is deposited on a Peltier plate with an initial air gap of 2.60 mm; it is then compressed at a constant rate of 100 μm/s to 70% of the initial air gap, at 25° C.; finally, the cohesivity of the gel is measured at the end of the compression course. The more a gel is cohesive, i.e., has a high cohesivity value, the more it is able to withstand stresses, such as those it may encounter after administration to a subject.

Preferentially, a hydrogel according to the present invention, acceptable for the therapeutic and/or cosmetic applications targeted by the present invention, has a tack test result ranging from 1.5 to 15 cm, preferentially from 1.5 to 10 cm, even more preferentially from 2 to 7.

Preferentially, a hydrogel according to the present invention acceptable for the therapeutic and/or cosmetic applications targeted by the present invention has a sticky character and will adhere to surfaces such as glass, metal, human tissues, collagen or plastic. In addition, it will maintain a cohesive structure as it moves over these surfaces.

### Composition

Another object of the present invention is a composition comprising a hydrogel according to the present invention. It is preferably a cosmetic or pharmaceutical composition. It may also comprise physiologically acceptable excipients.

The hydrogel according to the invention comprises a crosslinked polysaccharide. The composition may further comprise a non-crosslinked polysaccharide.

The composition according to the present invention can thus comprise from 0.1 to 5% by weight, preferably from 1 to 3% by weight of polysaccharide (e.g., hyaluronic acid), relative to the total weight of said composition, the polysaccharide (e.g., hyaluronic acid) being present in crosslinked and optionally non-crosslinked form. In particular, the content of non-crosslinked polysaccharide (e.g., hyaluronic acid) varies from 0 to 40% by weight, preferentially from 1 to 40% by weight, more preferentially from 5 to 30% by weight, relative to the total weight of polysaccharide (e.g., hyaluronic acid) present in the composition.

The polysaccharide of the hydrogel is as defined above.

The composition according to the present invention is preferably a sterile composition, especially heat sterilized at a plateau temperature comprised between 121° C. and 135° C., preferably for a plateau duration between 1 minute and 20 minutes with F0≤15.

It is preferably an injectable composition. The composition according to the invention then preferably comprises a physiologically acceptable medium, preferably a physiologically acceptable aqueous medium.

The physiologically acceptable aqueous medium may comprise a physiologically acceptable solvent or a mixture of solvents and preferably comprises water.

The physiologically acceptable medium may also comprise isotonic agents such as saccharides, sodium chloride and mixtures thereof.

The physiologically acceptable medium may also comprise at least one isotonic and physiologically acceptable saline solution.

Preferably, said equilibrated saline solution is a phosphate-buffered saline solution, and particularly a KH₂PO₄/K₂HPO₄ saline solution buffer.

The composition according to the invention may also comprise at least one additional compound chosen from lubricants, anesthetics, antioxidants, amino acids, vitamins, minerals, nucleic acids, co-enzymes, adrenaline derivatives, and mixtures thereof. The additional compounds can be as described above.

### Applications

The hydrogel or composition according to the invention can have therapeutic and/or cosmetic applications.

The present invention therefore also relates to a hydrogel or a composition according to the invention for its use in filling and/or replacing tissues, in particular soft tissues, especially by injecting the hydrogel or composition into the tissue.

In particular, the hydrogel or composition according to the invention is used in oral care and more particularly in the treatment of gingival recession, or to fill periodontal pockets. More particularly, the hydrogel or composition according to the invention is used to treat defects in the gingival architecture that can occur with tooth loss, with aging, with periodontal diseases and disorders, or after the installation of tooth implants, crowns or bridges.

The hydrogel or composition according to the invention can also be used in ophthalmology, more particularly to protect ocular structures during eye surgery such as, for example, ophthalmic surgery of the anterior or posterior segment, cataract extraction possibly with implantation of an intraocular lens, corneal transplantation surgery, glaucoma filtering surgery, or implantation of a secondary lens. In this case, the hydrogel or composition according to the invention will more particularly be injected into the eye.

The hydrogel or composition according to the invention can also be used in orthopedics or rheumatology, for example by injection into the synovial cavity. The hydrogel or composition according to the invention is then used as viscosupplementation.

The hydrogel or composition according to the invention can also be used in the treatment of lipodystrophy.

The hydrogel or composition according to the invention can be used in cosmetic surgery, in particular for gynecoplasties and/or penoplasties.

The hydrogel or composition according to the invention is administered more particularly by injection.

Advantageously, the hydrogel or composition according to the invention can be used for its mucoadhesive properties which are useful in the treatment of gingival pain.

The present invention also concerns a method for treating the pathologies indicated above, which comprises administering, to an individual in need thereof, an effective dose of the hydrogel or of the composition.

The effective dose of the hydrogel or of composition varies according to numerous parameters such as, for example, the chosen route of administration, weight, age, sex, the progression of the pathology to be treated and the sensitivity of the individual to be treated.

An object of the present invention is preferably the cosmetic, and therefore non-therapeutic, use of a hydrogel or of a composition according to the invention for preventing and/or treating the alteration of the viscoelastic or biomechanical properties of the skin, and, in particular, for stimulating, regenerating moisturizing, firming or restoring the radiance of the skin, especially by mesotherapy; for filling volume defects in the skin, and especially filling wrinkles, fine lines or scars (in particular hollow scars); or for reducing the appearance of wrinkles and fine lines.

For example, the present invention relates to the cosmetic use of a hydrogel or of a composition according to the invention for attenuating nasolabial folds and frown lines; for increasing the volume of the cheekbones, of the chin or of the lips; for restoring the volumes of the face, especially the cheeks, temples, the contour of the face, and of the periphery of the eye; or for stimulating, regenerating, moisturizing, firming or restoring the radiance of the skin, especially by mesotherapy.

In particular, the hydrogel or composition according to the invention is an anti-aging hydrogel or composition. The hydrogel or composition according to the invention is administered more particularly by injection.

The present invention also concerns a cosmetic, preferably anti-aging, treatment method for keratin materials, in particular the skin, comprising at least a step of administering a hydrogel or a composition according to the invention onto or through said keratin materials, more particularly by injection.

The administration may be an injection, in particular an intra-epidermal and/or intradermal and/or subcutaneous injection. The administration by intra-epidermal and/or intradermal and/or subcutaneous injection according to the invention aims to inject a hydrogel or a composition of the invention into an epidermal, dermoepidermal and/or dermal region. The hydrogel or composition according to the invention can also be administered by a supraperiosteal injection.

The hydrogel or composition according to the invention can be injected using any of the methods known to the person skilled in the art. In particular, a hydrogel or a composition according to the invention may be administered by means of an injection device suitable for an intra-epidermal and/or intradermal and/or subcutaneous and/or supraperiosteal injection.

The injection device may especially be chosen from a syringe, a set of microsyringes, a wire, a laser or hydraulic device, an injection gun, a needleless injection device, or a microneedle roller.

The injection device may have any injection means conventionally used suitable for intraepidermal and/or intradermal and/or subcutaneous and/or supraperiosteal injection.

Preferably, such a means may be a hypodermic needle or a cannula or a set of microneedles.

A needle or cannula according to the invention may have a diameter varying from 18 to 34 G, preferably between 25 and 32 G, and a length varying from 4 to 70 mm, and preferably from 4 to 25 mm. The needle or cannula is advantageously for single use.

Advantageously, the needle or cannula is associated with a syringe or any other device making it possible to deliver said hydrogel or said injectable composition through the needle or cannula.

According to a variant embodiment, a catheter may be interposed between the needle/cannula and the syringe. In a known manner, the syringe can be actuated manually by the practitioner or by a syringe holder such as a gun.

Preferably, the injection device is a syringe.

In a variant embodiment, the injection device can be adapted to the technique of mesotherapy.

Mesotherapy is a technique of treatment by intra-epidermal and/or intradermal and/or subcutaneous injection of a composition or hydrogel. The composition or hydrogel is administered according to this technique by injection in the form of multiple small droplets at the epidermis, the dermoepidermal junction and/or the dermis in order, especially to achieve a subcutaneous coating. The technique of mesotherapy is especially described in the book “Traité de mésotherapie [Treatise on mesotherapy” by Jacques Le Coz, Masson edition, 2004.

Mesotherapy done on the face is also called a mesolift or mesoglow.

Administration may also be topical.

Preferably, it is a topical application on the surface of the skin, more particularly on the epidermis, even more particularly on the facial epidermis.

The present invention thus also relates to an injection device as described above comprising a hydrogel or a composition according to the invention.

The examples which follow are given for illustrative purposes but should in no way be considered as limiting the present invention.

### Examples

### 1. Materials

1.5. MDa and 4 MDa non-crosslinked sodium hyaluronate (HTL),
BDDE (Sigma Aldrich),
DVS (Fisher Scientific),
0.25 M NaOH,
1 M HCl,
Phosphate buffer (Braun),
Lidocaine hydrochloride,
Three-dimensional stirrer,
DHR-2 rheometer,
Dynamometer and test bench,
Paddle mill homogenizer,
Sterile polyethylene bag.

### 2. Methods

### 2.1 Measuring the Viscoelastic Properties

The viscoelastic properties of the hydrogels obtained were measured using a rheometer (DHR-2) having a stainless-steel cone (1°-40 mm) with cone-plane geometry and an anodized aluminum Peltier plate (42 mm) (air gap 24 μm). 0.5 g of sterilized hydrogel is deposited between the Peltier plate and said cone. Then a stress scan is carried out at 1 Hz and 25° C. The elastic modulus G′, the viscous modulus G″ and the phase angle δ are reported for a stress of 5 Pa.

The stress at the intersection of G′ and G″, τ, is determined at the intersection of the curves of the G′ and G″ moduli and is expressed in Pascals.

### 2.2 Measuring the Extrusion Force

The extrusion forces (in Newtons) of the gels packaged in syringes were measured by means of a dynamometer-equipped test bench at a constant speed of 12.5 mm/min, through a 27 G, % inch needle and at room temperature. Extrusion force results are the average of the extrusion forces over at least 2 samples.

### 2.3 Identifying the Extensibility (Tack Test)

The tack test is conducted on a DHR-2 rheometer (TA instruments) equipped with a rough steel geometry (40 mm diameter) and a rough steel Peltier plate (40 mm diameter). 1 g of product is deposited between the two geometries at 25° C. and the upper geometry is rapidly brought closer to the Peltier plate at a speed of 2 mm/s up to an air gap of 2 mm. The upper geometry then compresses the gel at an approach speed of 0.1 mm/s up to an air gap of 0.1 mm. The upper geometry is then raised to 0.1 mm/s and the distance over which the gel maintains its extensible character between the 2 geometries is reported. The extensibility thus determined by a length unit is expressed in cm.

### 2.4 Measuring the Cohesivity

For the measurement of cohesivity (or mechanical strength, expressed in Newtons), the gel is deposited on a Peltier plate with an initial air gap of 2.60 mm. The gel is then compressed at a constant speed of 100 μm/s up to 70% of the initial air gap at 25° C. The cohesivity of the gel is measured at the end of the compression course.

### 2.5 Analyzing the Soluble Hyaluronic Acid

The soluble hyaluronic acid or sHA is analyzed using high performance liquid chromatography interfaced with a multiangle light scattering detector and a refractive index detector (HPLC-SEC-MALS-RI, ASTRA software (Wyatt Technology Corp.). The samples are diluted approximately 10 times according to their initial concentration of hyaluronic acid in the SEC mobile phase, a filtered solution of sodium nitrate 150 mM (pH 7.2). The diluted mixture is made up of compact and insoluble hyaluronic acid and soluble hyaluronic acid. The soluble hyaluronic acid portion of the samples was released for 5 days under orbital shaking to avoid artificial production of soluble hyaluronic acid.

The soluble part is separated from the insoluble part by a gentle filtration method (syringe equipped with a 0.45 μm filter) and then subjected to an SEC analysis.

Varying injection volumes are tested to achieve a background-free signal of at least five to one to prevent and avoid overloading the SEC columns.

The HPLC-SEC system uses a column duo for wide hyaluronic acid ranges from 500 Da to 20 MDa for optimal peak resolution on the chromatograph.

In order to have an optimal molecular weight analysis, a refractive increment index value or dn/dc (refractive index variation/polysaccharide concentration in the analytical solvent) of 0.165 mL/g was determined on the basis of internal samples analyzed under the same conditions as the samples.

Chromatograms obtained by SEC are analyzed to quantify the molecular weight, distribution and proportion of soluble hyaluronic acid in each sample.

The weight average molecular weight of the sample is data provided by the HPLC-SEC software. The percentage of soluble hyaluronic acid fractions (% sHA) is also a direct output from the HPLC-SEC software after entering the mass concentration (mg/mL) of total hyaluronic acid of the analyzed sample.

Said percentage may differ from one composition to another depending on its manufacturing technique and the dispersion (w/d) (weight molar mass/number molar mass) of the sample.

The % sHA for several molecular weight limits (<250 kDa, <100 kDa, <50 kDa, <30 kDa and <20 kDa) are also a direct output of the HPLC software. However, this does not take into account the quantity of hyaluronic acid actually released from the gel.

As a result, a normalized % sHA for multiple molecular weight limits was calculated to normalize the values provided by the software taking into account the quantity of hyaluronic acid actually released from the sample analyzed.

It is then possible to compare the results directly.

### 3. Examples

### Example 1: Hyaluronic Acid Gels Crosslinked with BDDE at Temperatures Either Below or Above the Freezing Point of the Reaction Medium

Prototypes No. 1 to 9 were prepared in the following manner with the BDDE contents (BDDE molar CR) described in Table 1.

BDDE and sodium hyaluronate (1.5 MDa, 120 mg/g) were dissolved in a 0.25 M aqueous sodium hydroxide solution in a sterile bag. The mixture was then homogenized in a paddle mill for 3 cycles of 15 min at 210 rpm at room temperature.

The mixture was then maintained at atmospheric pressure and placed at the temperatures and durations shown in Table 1. The pH of the mixture was approximately 13.

When the method was carried out at temperatures below the freezing point of the reaction medium (BDDE, sodium hyaluronate, aqueous sodium hydroxide solution), the mixture was left to thaw before continuing the preparation.

A 1 N HCl solution was then added to the sterile bag to obtain a pH of 7.3±0.5. The mixture was diluted to a concentration of 23 mg hyaluronic acid per gram of product with phosphate buffered saline (PBS). The mixture was homogenized for 24 h using a three-dimensional stirrer. The mixture was dialyzed.

Sodium hyaluronate (4 MDa) was added as a lubricant.

An aqueous solution of lidocaine hydrochloride was added to obtain 0.3% by weight of lidocaine hydrochloride relative to the weight of the resulting product.

The product thus obtained was sieved and then packaged in a syringe.

Finally, the product was sterilized by autoclave (plateau temperature between 121° C. and 135° C. with F0≤15).

After sterilization, the prototypes were analyzed. The elastic modulus G′, the phase angle δ and the stress at the intersection of G′ and G″, τ were determined. The results are presented in Table 1 below.

TABLE 1
Results

| | Cross-linking | | BDDE | | | | | |
|---|---|---|---|---|---|---|---|---|
| | T | Duration | molar CR | G′ | δ | τ | F | Cohesivity |
| Prototypes | (° C.) | (days) | (%) | (Pa) | (°) | (Pa) | (N) | (N) |
| 1 | 21 | 3 | 2.2 | 15 ± | 49.6 ± | N.D. | 7.9 ± | N/A |
| | | | | 2 | 6.7 | | 0.4 | |
| 2 | 21 | 3 | 6.2 | 173 ± | 12.3 ± | 465 ± | 9.7 ± | 8.6 |
| | | | | 5 | 0.3 | 41 | 0.1 | |
| 3 | −20 | 28 | 2.0 | 805 ± | 10.3 ± | 204 ± | 11.0 ± | 8.7 |
| | | | | 59 | 0.6 | 27 | 0.1 | |
| 4 | −20 | 61 | 1.0 | 1089 ± | 9.5 ± | 153 ± | 10.6 ± | 8.5 |
| | | | | 66 | 0.4 | 16 | 0.4 | |
| 5 | −20 | 32 | 1.0 | 281 ± | 18.2 ± | 369 ± | 12.7 ± | N/A |
| | | | | 11 | 0.5 | 6 | 0.3 | |
| 6 | −20 | 42 | 0.4 | 205 ± | 24.4 ± | 285 ± | 13.7 ± | 9.9 |
| | | | | 22 | 0.9 | 23 | 0.9 | |
| 7 | −20 | 120 | 0.4 | 956 ± | 9.9 ± | 139 ± | 9.8 ± | 7.7 |
| | | | | 63 | 0.6 | 36 | 0.3 | |
| 8 | −20 | 21 | 0.8 | 141 ± | 28.9 ± | 218 ± | 14.8 ± | 8.4 |
| | | | | 21 | 1.9 | 21 | 0.2 | |
| 9 | −20 | 31 | 0.8 | 218 ± | 23.3 ± | 328 ± | 13.8 ± | 9.5 |
| | | | | 14 | 0.7 | 15 | 0.3 | |

For reference, the properties of a commercial product, Restylane Lyft, which has a degree of modification of 1% (lowest known value of crosslinked hyaluronic acid gels for aesthetic application), are presented in Table 2.

TABLE 2
Properties of a commercial product

| | G′ | δ | τ | F | Cohesivity |
|---|---|---|---|---|---|
| | (Pa) | (°) | (Pa) | (N) | (N) |
| Restylane Lyft | 855 ± 10 | 6.8 ± 0.1 | 43 ± 1 | N.D. | 2.2 |

The commercial product, Restylane Lyft, has a very low cohesivity (2.2 N) compared with the gels obtained by means of the method of the present invention.

While cross-linking for 3 days at 21° C. with a low molar cross-linking rate of 2.2% did not allow the preparation of a gel (prototype 1 with 6 greater than 45°), the method of the present invention made it possible to prepare hydrogels with desirable mechanical properties with a molar cross-linking rate of only 0.4% (prototype 6).

When the cross-linking time is increased, the elastic modulus increases, but the gel has a lower τ. In other words, it is potentially more brittle (see prototypes 5 vs. 4 and 7 vs. 6).

Surprisingly, the gels obtained by the method of the present invention possess rheological properties and cohesivities which are at least as interesting, or even more (the HA concentrations and the manufacturing method being otherwise identical), than the gels obtained conventionally at room temperature and with a markedly higher degree of cross-linking (prototype 2).

### Example 2: Method According to the Invention for Hyaluronic Acid Crosslinked to DVS

Prototypes X1 and X2 were prepared in the following manner with the DVS contents (DVS molar CR) described in Table 3.

Sodium hyaluronate (1.5 MDa, 120 mg/g) is dissolved in a 0.25 M aqueous solution of DVS and sodium hydroxide in a sterile bag. The mixture is homogenized in a paddle mill for 3 cycles of 15 min at 210 rpm at room temperature. The mixture is placed and maintained at the temperatures and times shown in Table 3. The pH of the mixture was approximately 13.

The mixture is left to thaw before continuing preparation.

A 1 N HCl solution is added to the sterile bag until a pH of 7±0.5 is obtained. 6—the mixture is diluted to a concentration of 23 mg hyaluronic acid per gram of product with phosphate buffer PBS.

The mixture is homogenized for 24 h by means of a three-dimensional stirrer.

The mixture is then dialyzed and a fixed quantity of sodium hyaluronate (4 MDa) is added as lubricant.

An aqueous solution of lidocaine hydrochloride is added to obtain 0.3% by weight of lidocaine hydrochloride relative to the weight of the resulting product.

The product thus obtained was sieved and then packaged in a syringe.

Finally, the product was sterilized by autoclave (plateau temperature between 121° C. and 135° C. with F0≤15).

The prototype X0 (comparative) was prepared identically to the prototype X1. Only the cross-linking step was changed, i.e., 0.75 day of cross-linking at 21° C.

TABLE 3
cross-linking conditions

| | DVS molar CR | Temperature | Duration |
|---|---|---|---|
| Prototypes | (%) | (° C.) | (days) |
| X0 | 0.3 | 21 | 0.75 |
| X1 | 0.3 | −20 | 15 |
| X2 | 1.3 | −20 | 15 |

After sterilization, the prototypes were analyzed. The elastic modulus G′, the phase angle δ and the stress at the intersection of G′ and G″, τ were determined. The results are presented in Table 4.

TABLE 4
Results

| Prototypes | G′ (Pa) | δ (°) | τ (Pa) |
|---|---|---|---|
| X0 | 56 ± 9 | 47.7 ± 2.5 | N/A |
| X1 | 140 ± 34 | 34.0 ± 1.6 | 188 ± 21 |
| X2 | 433 ± 64 | 15.8 ± 3.1 | 147 ± 14 |

The prototypes of hyaluronic acid crosslinked to DVS, X1 and X2, are gels (phase angles less than 45° C.) and have properties suitable for the therapeutic, cosmetic and aesthetic applications targeted by the present invention (stress at the intersection of G′ and G″ greater than 50 Pa and G′ greater than 20 Pa).

The prototype X0 produced with the same degree of cross-linking as X1 but conventionally (cross-linking at room temperature) does not appear in the form of a gel (δ>45°).

The method of the present invention appears to be well suited to cross-linking agents possessing chemical functions other than epoxides.

### Example 3: Stability Study of Cryogels According to the Invention

The prototypes presented in Table 5 were placed in an accelerated and forced degradation oven at 40° C. to monitor the evolution of the rheological properties over a period of 6 months.

These conditions model storage equivalent to 2 years at room temperature. Each prototype was produced according to the synthesis protocol described in Example 1 with the conditions presented in Table 5.

TABLE 5
cross-linking conditions

| | BDDE molar | | |
|---|---|---|---|
| | cross-linking | | |
| | rate | Temperature | Duration |
| Prototypes | (%) | (° C.) | (days) |
| Y1 | 2.1 | −20 | 28 |
| Y2 | 1.0 | −20 | 61 |
| Y3 | 0.4 | −20 | 30 |
| Y4 | 6.2 | 21 | 3 |

The change in phase angle has been measured over time and is presented in Table 6 as phase angle loss: % phase angle loss=(phase angle at 6 months at 40° C.—initial phase angle)/initial phase angle*100

TABLE 6
Results

| | % phase angle |
|---|---|
| Prototypes | loss |
| Y1 | 11.7 |
| Y2 | 20.2 |
| Y3 | 14.1 |
| Y4 | 18.3 |

The prototypes obtained by the method of the present invention, Y1, Y2, and Y3, with low cross-linking rates show a small loss of their viscoelastic properties after an accelerated aging study mimicking 2 years of storage at 25° C. in an unfavorable situation. This loss is acceptable and similar to that of a traditional gel (Y4) obtained by a conventional method (cross-linking at room temperature) with a higher molar cross-linking rate.

### Example 4: Analysis of the Soluble Hyaluronic Acid of the Cryogels

The soluble hyaluronic acid of the gels of Table 7 (gels prepared according to Example 1) was analyzed by HPLC-SEC-equipped with MALS and RI detectors. The gels after sterilization were diluted with gentle stirring at 37° C. to extract soluble hyaluronic acid. The results are presented in Table 8. The weight average molecular weight (MW) and the quantity of soluble hyaluronic acid with MW less than 50 kDa are listed.

TABLE 7
cross-linking conditions

| | BDDE molar | | |
|---|---|---|---|
| | cross-linking | | |
| | rate | Temperature | Duration |
| Prototypes | (%) | (° C.) | (days) |
| K1 | 2.2 | 21 | 3 |
| K2 | 6.2 | 21 | 3 |
| K3 | 2.0 | −20 | 28 |
| K4 | 1.0 | −20 | 61 |
| K5 | 0.4 | −20 | 42 |

TABLE 8
Results

| | | sHA |
|---|---|---|
| | | normalized |
| | | content <50 |
| | | kDa (% |
| | | hyaluronic |
| | | acid per |
| Prototypes | MW (kDa) | syringe) |
| K1 | 797 | 0.3 |
| K2 | 510 | 0.8 |
| K3 | 928 | 0.0 |
| K4 | 808 | 0.0 |
| K5 | 706 | 0.0 |

All these prototypes are formulated with identical raw materials of hyaluronic acid with an MW greater than 1 MDa. The production of fragments below 50 kDa thus indicates a certain degree of degradation of hyaluronic acid during the process.

Thus, it is clear that the 2 prototypes (K1 and K2) obtained conventionally are the only prototypes with traces of soluble hyaluronic acid with an MW of less than 50 kDa. All the prototypes obtained by the method of the invention, any other parameter being otherwise equal, exhibit better preservation of the integrity of the hyaluronic acid chains and therefore no trace of sHA less than 50 kDa.

### Example 5: Comparing the Integrity of Soluble Hyaluronic Acid Chains of Cryogels Obtained by a Method According to the Invention or by a Freezing Method According to the Prior Art

The size distribution of hyaluronic acid chains in a hydrogel prepared according to the method described in Oschlaeger C.; Bossler, F.; Willenbacher, N. Synthesis, structural and micro-mechanical properties of 3D hyaluronic acid-based cryogel scaffolds Biomacromolecules 2016), and according to the invention, was compared in order to evaluate the respective capacity of each method to preserve hyaluronic acid chains.

According to the method of the prior art, a first step of dissolving the hyaluronic acid fibers in the sodium hydroxide is carried out over 24 h at 4° C. Since the cross-linking process has already begun during these 24 h before cryogelation, a cryogelation step of only 10 days is necessary in order to obtain gels with interesting properties close to those of the present invention.

In the method of the present invention, this step of dissolving the hyaluronic acid fibers (step a); preparing the reaction medium) is carried out at room temperature over about 1 h followed by freezing over a much longer period. The conditions of this comparison are summarized in Table 9.

In order to compare the impact of each method on the integrity of the hyaluronic acid chains, hyaluronic acid fibers were subjected to both methods without the use of a cross-linking agent. Thus, after each key step of the methods (fiber dilution, cryogelation), it is possible to follow MW and hyaluronic acid chain distribution by HPLC-SEC. A decrease in MW and an increase in the presence of small fragments of hyaluronic acid are then synonymous with the degradation of hyaluronic acid by the method. For each HPLC-SEC analysis (system described above), samples were neutralized to physiological pH and diluted to approximately 0.5 mg/mL.

TABLE 9
Summary of the 2 methods compared

| | Method according to | Method according |
|---|---|---|
| | the prior art | to the invention |
| Initial MW | 859 kDa | 859 kDa |
| of hyaluronic | | |
| acid fibers used | | |
| Dissolution of | 24 h at 4° C. at 60 | 1 h at 21° C. at 60 |
| fibers | mg/mL hyaluronic acid | mg/mL hyaluronic acid |
| | concentration in 1% | concentration in 1% |
| | NaOH | NaOH |
| Cryogelation | 10 days at −20° | 30 days at −20° |
| | C., at 60 mg/mL | C., at 60 mg/mL |
| | hyaluronic acid concen- | hyaluronic acid concen- |
| | tration in 1% NaOH | tration in 1% NaOH |
| Neutralization | After the fiber dissolution | After the fiber dissolution |
| for HPLC-SEC | step (24 h) (sample A1) and | step (1 h) (sample C), |
| sample | after cryogelation for 10 | after 24 h of cryogelation |
| preparation | days (sample A2) with 1M | (sample B1 compared to |
| | HCl and dilution to 0.5 | 24 h with Oeschlaeger |
| | mg/mL with phosphate | et al.) and after |
| | buffer | cryogelation for 30 days |
| | | (sample B2) with 1M HCl |
| | | and dilution to 0.5 mg/mL |
| | | with phosphate buffer |
| MW of each | A1: 734 kDa | C: 838 kDa |
| sample | A2: 737 kDa | B1: 843 kDa |
| | | B2: 841 kDa |

Starting from the same raw material with an MW of 859 kDa and using the same aqueous solutions for dissolution, neutralization and dilution as well as using the same equipment and the same conditions for analyzing hyaluronic acid chains, we were able to observe that the method according to the prior art inflicts a loss of about 100 kDa on the MW of hyaluronic acid compared to the method of the present invention. This is also reflected in the presence of 0.5% hyaluronic acid with an MW<50 kDa whereas the method of the present invention does not present any trace of these hyaluronic acid fragments.

It is interesting to note that the freezing steps of the 2 methods do not increase the degradation of hyaluronic acid since the only degradation observed takes place during the fiber dissolution step over 24 h at 4° C. Thus only this step has an impact on the MW of hyaluronic acid. The control sample (dissolution 1 h at 21° C. according to the method of the present invention) showed no degradation compared to the initial raw material. A decrease in MW will have a negative impact on the final viscoelastic properties of the gels and the presence of small fragments of hyaluronic acid may have an impact on local biological reactions of inflammation after implantation of the gel in vivo.

### Example 6: Evaluating the Extensibility of the Gels (Tack Test)

The extensibility of the gels presented in Table 10 (prepared according to Example 1) was evaluated by the tack test by measuring the distance over which the gel is capable of maintaining a bond/cohesivity between 2 surfaces.

TABLE 10
Cross-linking conditions

| | BDDE molar | | |
|---|---|---|---|
| | cross-linking | | |
| | rate | Temperature | Duration |
| Prototypes | (%) | (° C.) | (days) |
| Z1 | 2.2 | 21 | 3 |
| Z2 | 6.2 | 21 | 3 |
| Z3 | 0.4 | −20 | 42 |
| Z4 | 2.2 | −20 | 32 |

The tack test data are presented in Table 11.

TABLE 11
Results

| | BDDE molar | | | | | |
|---|---|---|---|---|---|---|
| | cross-linking | | | | | Tack |
| | rate | G′ | Phase | τ | Cohesivity | test |
| Prototypes | (%) | (Pa) | angle (°) | (Pa) | (N) | (cm) |
| Z1 | 2.2 | 15 ± 2 | 49.6 ± 6.7 | N/A | N/A | 3.1 ± 0.1 |
| Z2 | 6.2 | 173 ± 5 | 12.3 ± 0.3 | 465 ± 41 | 8.6 | 1.3 ± 0.2 |
| Z3 | 0.4 | 205 ± 22 | 24.4 ± 0.9 | 285 ± 23 | 9.9 | 4.2 ± 0.5 |
| Z4 | 2.2 | 1350 ± 72 | 10.3 ± 0.4 | 176 ± 20 | N/A | 0.9 ± 0.1 |

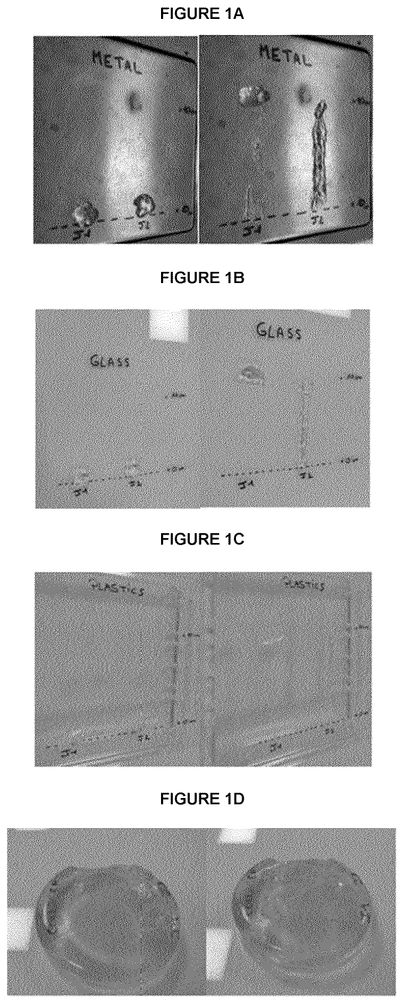

FIG. 1 illustrates the implementation of the tack test on the prototypes Z2 and Z3. After compression of the gel, the surfaces are separated.

Prototypes Z1 and Z3 have the highest extensibility. Z1, however, according to its rheological properties, is not a viscoelastic gel (phase angle >45°) and therefore behaves as a viscous solution.

Z3, on the contrary, is indeed a gel that has a greater range of stretch than other prototypes. Z2 and Z4 do not have an extensible character. Z2 is a gel obtained conventionally (cross-linking at room temperature) while Z4 is a hydrogel obtained by the method of the present invention, with a very high molar cross-linking rate (>2%), which therefore makes the gel simultaneously very elastic, brittle and non-extensible.

Thus, the Z3 prototype obtained by the method of the present invention with a low modification rate gives the best results in terms of obtaining a viscoelastic gel with desirable properties and an extensible gel (cohesivity in traction).

### Example 7: Evaluation of Stickiness, Affinity of Gels with Surfaces

The stickiness of a gel according to the invention J2 (prototype 6 of Example 1) and of a conventional gel J1 (prototype 2 of Example 1) was observed qualitatively. The gels are presented in Table 12. The adhesion or non-adhesion of the gel tested to a model surface is observed after depositing a bolus of 1 g of gel on said surface. The gel is pushed 10 cm and the state of the bolus and behavior of the gel are observed. The four model surfaces are a metal surface (FIG. 1A), glass surface (FIG. 1B), plastic surface (FIG. 1C), and a gelatin surface (FIG. 1D).

TABLE 12
Cross-linking conditions

| | BDDE molar | | |
|---|---|---|---|
| | cross-linking | | |
| | rate | Temperature | Duration |
| Prototypes | (%) | (° C.) | (days) |
| J1 | 6.2 | 21 | 3 |
| J2 | 0.4 | −20 | 42 |

The prototype J1 obtained by a conventional cross-linking technique tends to roll on the model surfaces when trying to move it. On the contrary, the prototype J2, obtained by cryogelation and having a G′ relatively close to J1, tends to adhere systematically to these four model surfaces.

The force required to move J2 is also greater on the four model surfaces due to the adhesion of the gel over its movement. It should also be noted that despite the adhesion of the prototype J2 over its path of movement, it maintains its cohesivity (a single block of gel) and does not crumble along its movement on the surfaces. This can be an important advantage in vivo because the gel will tend to migrate less by adhering to surfaces.

1. A method for preparing a hydrogel comprising the following steps:
a) providing at least one polysaccharide;
b) providing at least one crosslinking agent, said crosslinking agent comprising at least two functional groups Z, identical or different, chosen from isocyanate, amino, epoxide, carboxyl, N-succinimidyloxycarbonyl, N-sulfosuccinimidyloxycarbonyl, halocarbonyl, isothiocyanate, vinyl, formyl, hydroxyl, sulfhydryl, hydrazino, acylhydrazino, aminoxy, carbodiimide, and an acid anhydride residue;
c) preparing a crosslinking reaction medium comprising the polysaccharide(s), the cross-linking agent(s) and a solvent, the total quantity of cross-linking agent ranging from 0.001 to less than 0.02 mole per 1 mole of polysaccharide repeating unit, the duration of the reaction medium preparation step not exceeding 5 hours;
d) placing the reaction medium obtained at the end of step c), for a period ranging from 2 weeks to 17 weeks, at a pressure P less than or equal to atmospheric pressure and at a temperature T greater than the eutectic point temperature of the reaction medium as measured at pressure P and less than the freezing point temperature of the reaction medium as measured at pressure P.

2. The method according to claim 1 wherein the polysaccharide is a glycosaminoglycan.

3. The method according to claim 1, wherein the functional groups Z of the cross-linking agent are epoxide groups.

4. The method according to claim 1 wherein the total quantity of cross-linking agent ranges from 0.001 to 0.01 moles per 1 mole of polysaccharide repeating unit.

5. The method according to claim 1 wherein the temperature T is greater than or equal to −55° C. and less than or equal to −5° C.

6. The method according to claim 1 wherein the pressure P is equal to atmospheric pressure.

7. The method according to claim 1 further comprising a sterilization step.

8. A hydrogel obtainable by the method of claim 1.

9. A cosmetic or pharmaceutical composition comprising a hydrogel according to claim 8 and a physiologically acceptable excipient.

10. A method for filling and/or replacing tissues comprising administering to a subject the hydrogel according to claim 8 or a composition comprising the hydrogel of claim 8 and a physiologically acceptable excipient.

11. A cosmetic method for for preventing and/or treating the alteration of viscoelastic or biomechanical properties of the skin; for filling in volume defects of the skin; to reduce nasolabial folds and frown lines, to increase the volume of the cheekbones, chin or lips, to restore facial volume; or to reduce the appearance of wrinkles and fine lines; or to stimulate, regenerate, moisturize, firm or restore the radiance of the skin, comprising administering to a subject the hydrogel according to claim 8 or a composition comprising the hydrogel of claim 8 and a physiologically acceptable excipient.

12. The method according to claim 1, wherein the hydrogel is injectable.

13. The method according to claim 2, wherein the polysaccharide is hyaluronic acid or a salt thereof.

14. The method according to claim 3 wherein the cross-linking agent is chosen from 1,4-butanediol diglycidyl ether (BDDE), 1,2,7,8-diepoxy-octane, poly(ethylene glycol) diglycidyl ether (PEGDG), 1,2-bis(2,3-epoxypropoxy)ethane (EGDGE), and their mixtures

15. The method according to claim 4 wherein the total quantity of cross-linking agent ranges from 0.001 to 0.005 moles per 1 mole of polysaccharide repeating unit.
